(19) United States
(12) Patent Application Publication
(10) Pub. No.: US 2009/0093826 A1
(43) Pub. Date: Apr. 9, 2009

(54) Patent Foramen Ovale Closure System

(75) Inventor: Zachary Warder-Gabaldon, Palo Alto, CA (US)

Correspondence Address: CARDICA, INC., 900 SAGINAW DRIVE, REDWOOD CITY, CA, 94063, US

(73) Assignee: CARDICA, INC., Redwood City, CA (US)

(21) Appl. No.: 11/868,431

(22) Filed: Oct. 5, 2007

(51) Int. Cl. A61B 17/08

(52) U.S. Cl. 606/151; 606/213

(57) ABSTRACT

A tool for closing an opening in tissue may include a catheter, a clasp applicator positioned substantially at the distal end of the catheter; and at least one clasp held by the clasp applicator, where the clasp may include a plurality of tines and be plastically splayable by the clasp applicator. A patent foramen ovale in heart tissue may by closed by, for example, advancing that exemplary clasp applicator through the patient's vasculature to a location in proximity to the patent foramen ovale, anchoring the clasp applicator with respect to the patent foramen ovale, splaying the clasp, advancing the splayed clasp into contact with tissue; closing the clasp, and removing the clasp applicator from the heart.

### FIELD OF THE INVENTION

The present invention relates generally to a system for closing a patent foramen ovale in heart tissue.

### BACKGROUND

Referring to FIG. 1, a patent foramen ovale (PFO) 2 is a flap-like opening in the wall 4 between the left atrium 6 and the right atrium 8 of the heart 10. In some instances, a PFO 2 may have a more tunnel-like configuration between the atria 6, 8. The PFO 2 typically closes at or shortly after birth. However, in an estimated 20-25% of people, the PFO 2 remains open into adulthood. The PFO 2 allows blood clots in the bloodstream to bypass the natural filtering mechanism of the lungs, which can lead to stroke. A person with a PFO 2 is generally asymptomatic, and generally does not know he or she has a PFO 2 until after a stroke. In addition, research suggests a correlation between PFO 2 and migraine, the mechanism of which is still unknown.

Currently, patients having a known PFO 2 are treated in one of two ways. The patient may be prescribed blood thinners such as coumadin, to reduce the risk of clot formation. However, this course of treatment requires lifelong dependence on that medication, which does nothing to close the opening between the atria. Alternately, a device may be placed through the PFO 2 and then unfurled on each side of the PFO 2, such that each end of the PFO 2 is covered by an unfurled part of the device. Such a device may include a material such as polyester stretched over a wire frame, or stuffed inside a wire mesh. However, such devices can expose a significant amount of metal or other material to the bloodstream, which is considered undesirable.

### BRIEF DESCRIPTION OF THE DRAWINGS

FIG. 1 is a cross-section view of a heart.

FIG. 2 is a schematic view of an exemplary PFO closure system.

FIG. 3 is a cutaway perspective view of an exemplary clasp applicator.

FIG. 4 is a perspective view of an exemplary clasp, deployable by the clasp applicator of FIG. 3, in an initial configuration.

FIG. 5 is a perspective view of another exemplary clasp in an initial configuration.

FIG. 6 is a perspective view of the exemplary clasp of FIG. 5 in a second, splayed configuration.

FIG. 7 is a cutaway perspective view of an exemplary clasp applicator.

FIG. 8 is a top view of an exemplary anvil assembly that may be a component of the clasp applicator.

FIG. 9 is a perspective view of exemplary anchors extending from an exemplary clasp applicator.

FIG. 10 is a perspective view of an anchor configured as a basket.

FIG. 11 is a side view of a different exemplary anchor configured as a basket, where the basket is in an initial configuration.

FIG. 12 is a side view of the basket of FIG. 11 in an expanded configuration.

FIG. 13 is a perspective view of anchors configured as harpoons, extending from the clasp applicator.

FIG. 14 is a top cutaway view of an exemplary harpoon of FIG. 13 in an initial configuration.

FIG. 15 is a top cutaway view of an exemplary harpoon of FIG. 13 in a deployed configuration.

FIG. 16 is a top cutaway view of an exemplary harpoon of FIG. 13 in a withdrawal configuration.

FIG. 17 is a cutaway view of the heart showing a step in a method of closing a patent foramen ovale.

FIG. 18 is a cutaway view of the heart showing another step in a method of closing a patent foramen ovale.

FIG. 19 is a cutaway view of the heart showing another step in a method of closing a patent foramen ovale.

FIG. 20 is a cutaway view of the heart showing another step in a method of closing a patent foramen ovale.

FIG. 21 is a cutaway view of the heart showing another step in a method of closing a patent foramen ovale.

FIG. 22 is a cutaway perspective view of an exemplary clasp applicator, showing a splayed clasp.

FIG. 23 is a perspective view of an exemplary clasp applicator, showing a splayed clasp.

FIG. 24 is a perspective view of the clasp of FIG. 4 in a closed configuration.

FIG. 25 is a perspective view of a patent foramen ovale closed by a clasp.

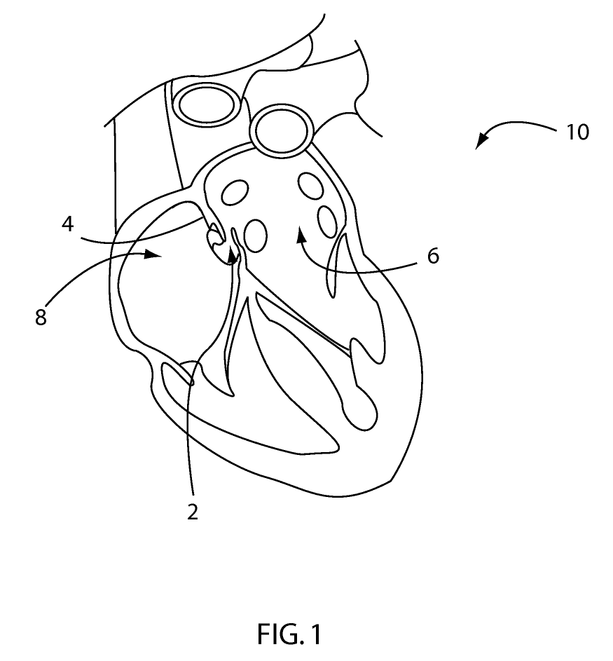

The use of the same reference symbols in different figures indicates similar or identical items.

### DETAILED DESCRIPTION

U.S. patent application Ser. No. 11/158,414, filed on Jun. 22, 2005, is hereby incorporated by reference in its entirety.

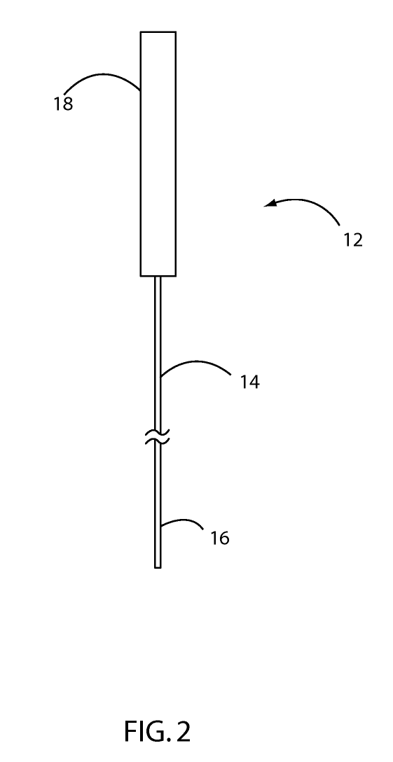

Referring to FIG. 2, a PFO closure system 12 includes a catheter 14 attached to and extending proximally from a clasp applicator 16. Alternately, the clasp applicator 16 is attached to a flexible rod, guidewire, or other suitable structure. A handle 18 is operationally connected to the clasp applicator 16. Where the clasp applicator 16 is connected to the catheter 14, the handle 18 may be connected to the catheter 14 as well. The handle 18 may be connected to the clasp applicator 16 by one or more cables, tubes, wires or any other suitable structures or mechanisms configured for passage through the lumen of the catheter 14. The clasp applicator 16 may be fixed to the handle 18, or may be detachable therefrom to allow reloading of a new clasp applicator 16 onto the handle 18 after use of a previous clasp applicator 16. The handle 18 may include any mechanism, mechanisms, structure or structures configured to actuate the clasp applicator 16. The handle 18 may also include a source of stored energy for actuating the clasp applicator 16. The source of stored energy may be mechanical (such as a spring), electrical (such as a battery), pneumatic (such as a cylinder of pressurized gas) or any other suitable source of stored energy. The source of stored energy, its regulation, and its use in actuating the clasp applicator 16 may be as described in the U.S. patent application Ser. No. 11/054,265, filed on Feb. 9, 2005, which is herein incorporated by reference in its entirety. The handle 18 may instead, or also, include a connector or connectors suitable for receiving stored energy from an external source, such as a hose connectable to a hospital utility source of pressurized gas or of vacuum, or an electrical cord connectable to a power source.

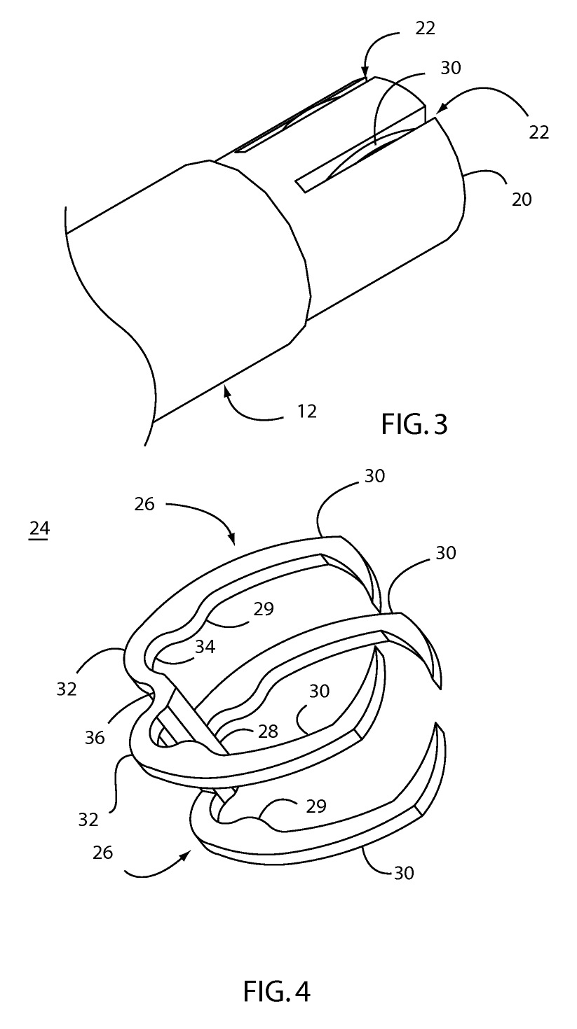

Referring also to FIG. 3, a clasp applicator 16 includes a housing 20. The housing 20 may be substantially tubular. Alternately, the housing 20 may be shaped in any other suitable manner. The housing 20 may be fabricated from any suitable material. At least one clasp 24 is held by the clasp applicator 16. One or more slots 22 may be defined in the housing 20. Advantageously, each slot 22 is oriented substantially longitudinally. Alternately, each slot 22 may be oriented in any suitable manner. Each slot 22 is sized and configured to allow at least part of the clasp 24 to splay outward through the slot 22, as described in greater detail below. The clasp applicator 16 may be fixed at least temporarily to the distal end of a catheter 14, where that catheter 14 may have any suitable number of lumens defined therethrough. Alternately, the clasp applicator 16 may be fixed at least temporarily to a different part of the catheter 14. Alternately, the clasp applicator 16 may be fixed at least temporarily to a guidewire, whether that guidewire is received by the catheter 14 or is utilized without a catheter 14.

Referring to FIG. 4, one example of a clasp 24 is shown. That exemplary clasp 24 includes two or more clips 26 affixed together by at least one backbone 28. Advantageously, each clip 26 may be a staple substantially as disclosed in U.S. patent application Ser. No. 11/093,003, “Vascular Closure System,” filed on Mar. 28, 2005 (the “'003 application”), or substantially as disclosed in U.S. patent application Ser. No. 11/282,177, “Vascular Closure System Utilizing a Staple,” filed on Nov. 4, 2005 (the “'177 application”), both of which are hereby incorporated by reference in their entirety. However, each clip 26 may be sized and shaped in any suitable manner. As one example, at least one clip 26 may be curved and generally M-shaped or W-shaped. However, at least one clip 26 may have any other suitable shape. At least one clip 26 may have a different shape than at least one other clip 26. At least one clip 26 may have two tines 30, each extending at least partially in the distal direction. The tines 30 may be curved, and may each have a shape and radius of curvature such that the tines 30 are generally not parallel to one another. The radius of curvature may be substantially coincident with the path of travel of the tines 30 during closure of the clasp 24. At least one clip 26 may be substantially bilaterally symmetrical, although it may be asymmetrical if desired. At least one clip 26, and the clasp 24 as a whole, may be a substantially continuous solid. Alternately, at least one clip 26 and/or the entire clasp 24 is not a substantially continuous solid. At least one clip 26 may lie substantially in a single plane. That is, at least one clip 26 may be shaped such that a single plane extends through and substantially bisects the entire clip 26. The clips 26 of the clasp 24 may be substantially parallel to one another. Alternately, the clips 26 may be oriented differently relative to one another.

At least one tine 30 may have a substantially pointed or sharpened distal end. However, the distal ends of the tines 30 need not be pointed or sharpened, particularly if the cross-sectional area of each tine 30 is small. Advantageously, each tine 30 has a single distal end. However, at least one tine 30 may have a distal end that is bifurcated, forked, split or otherwise configured. The body of an exemplary clip 26 extends proximally from the distal end of one tine 30 and curves or angles outward from the longitudinal centerline of the clip 26, then toward the longitudinal centerline of the clip 26. Alternately, the tine 30 may curve differently. The body of the clip 26 reaches a peak 32, then extends distally and toward the longitudinal centerline of the clip 26. A valley 34 is the area on the clip 26 on the other side of the clip 26 from a peak 32. For example, where a peak 32 of the clip 26 includes a convex curve oriented proximally, the corresponding valley 34 is a concave curve opening distally. The body of the clip 26 then reaches a trough 36, then extends proximally and away from the longitudinal centerline of the staple to a second peak 32. The body of the clip 26 continues distally to form the second tine 30, and ends at the distal end of the second tine 30. Optionally, a splay bump 29 may be positioned on an inner surface of at least one tine 30. The splay bump 29 facilitates splaying of the clip 26, as described in greater detail below. Alternately, the clip 26 may be shaped differently.

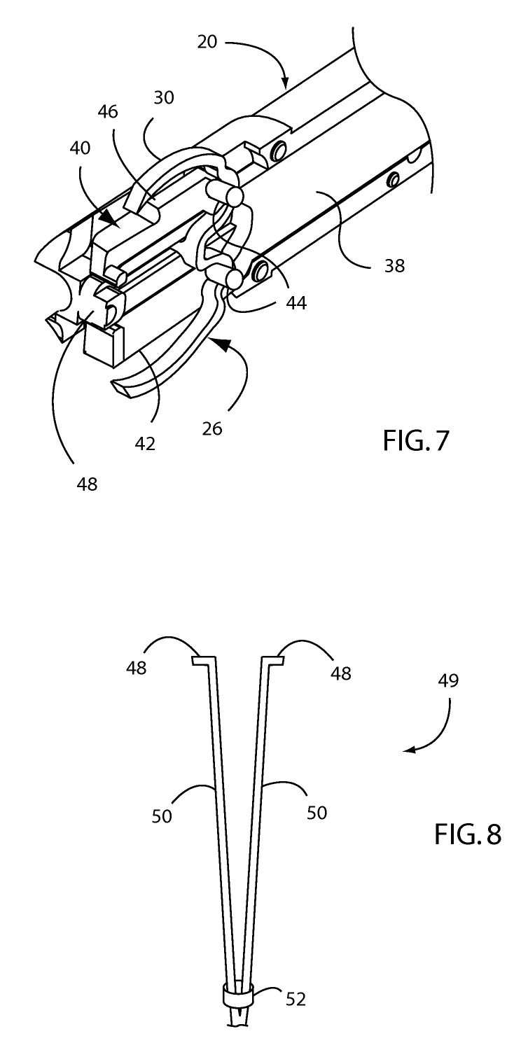

The backbone 28 may be a substantially straight bar, or may have any other suitable configuration. For example, the backbone 28 may be curved, may have a complex shape, or may include two or more struts or other structural elements. Where at least one clip 26 generally lies in a plane, the backbone 28 may be oriented substantially perpendicular to that plane, or may be oriented at any other angle or in any other suitable direction. The backbone 28 may be flexible, rigid, or configured in any other suitable manner. The backbone 28 can be made of the same material as the clips 30, or of any other suitable material, such as suture or wire. The backbone 28 may be affixed to any suitable portion of each clip 26. As one example, the backbone 28 may be affixed to the trough 36 of each clip 26. The backbone 28 may be fabricated integrally with the clips 26 such that the clasp 24 is fabricated as a single unit. Alternately, the clips 26 are fabricated individually, then attached to a backbone 28 such as by welding, soldering, or other suitable method. Alternately, the clasp 24 may be fabricated in any other suitable manner. Optionally, the backbone 28 may be frangibly connected to one or more components of the clasp applicator 16, such as the driver 38 of FIG. 7.

Advantageously, each clip 26 may be plastically deformable. If so, the clip 26 or entire clasp 24 may be fabricated from stainless steel, titanium or any other suitable plastically-deformable material. Alternately, at least one clip 26 may be elastically deformable. If so, such clip 26 may be fabricated from nickel-titanium alloy or any other suitable elastic or superelastic material. The cross-section of each clip 26 may vary at different locations along the clip 26, or may be substantially constant along the entire clip 26. For example, the cross-sectional area of each clip 26 at certain locations may be less than at other locations, in order to promote bending in those locations having a lesser cross-sectional area.

Referring also to FIG. 3, the clasp 24 initially may be positioned completely within a space inside the clasp applicator 16. Alternately, the clasp 24 may be positioned differently within the clasp applicator 16, or may be positioned at least partially outside the clasp applicator 16 rather than within it. The clasp 24 may be held by the clasp applicator 16 in any suitable manner. The clasp applicator 16 may hold more than one clasp 24, if desired.

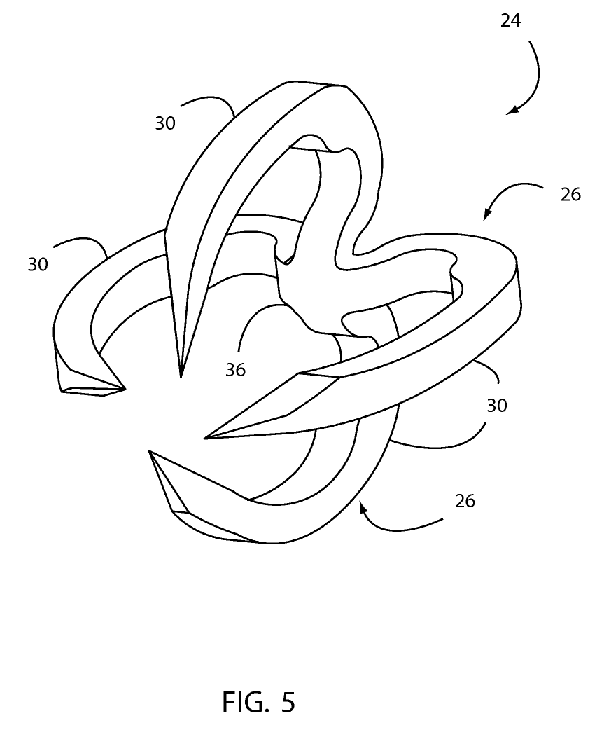

Referring to FIG. 5, another exemplary embodiment of a clasp 24 is shown, in a first, initial position. Advantageously, the clasp 24 is fabricated substantially as two clips 26, each substantially as disclosed in the '003 application or the '177 application, oriented at substantially right angles to one another and connected at the trough 36 of each staple. Alternately, the two clips 26 may be oriented at a different angle relative to one another. Alternately, such a clasp 24 may be fabricated substantially as three or more clips 26 connected together. The junction of the troughs 36 of the clips 26 may be characterized as the trough 36 of the clasp 24. Viewed on end, the clasp 24 may be generally X-shaped. However, the clasp 24 may be shaped differently. The clasp 24 may be fabricated integrally, or may be fabricated from two or more individual clips 26 that subsequently are fixed together. That is, each set of two opposed tines 30 of the clasp 24 may be shaped the same as or similar to the staples disclosed in the '003 application or the '177 application. Alternately, where the clasp 24 includes an odd number of tines 30, each tine 30 may be shaped the same as or similar to one-half of the staple disclosed in the '003 application or the '177 application. Alternately, at least one tine 30 may be shaped differently. The clasp 24 may include four tines 30. Alternately, three tines 30, or more than four tines 30, may be used. The clips 26 may be shaped substantially as described above relative to FIG. 4. The description of pairs of tines of the clasp 24 in this document as clips 26 is solely for convenience and for clarity of description, and does not require independent or frangible clips 26. Advantageously, the clasp 24 is a single one-piece structure.

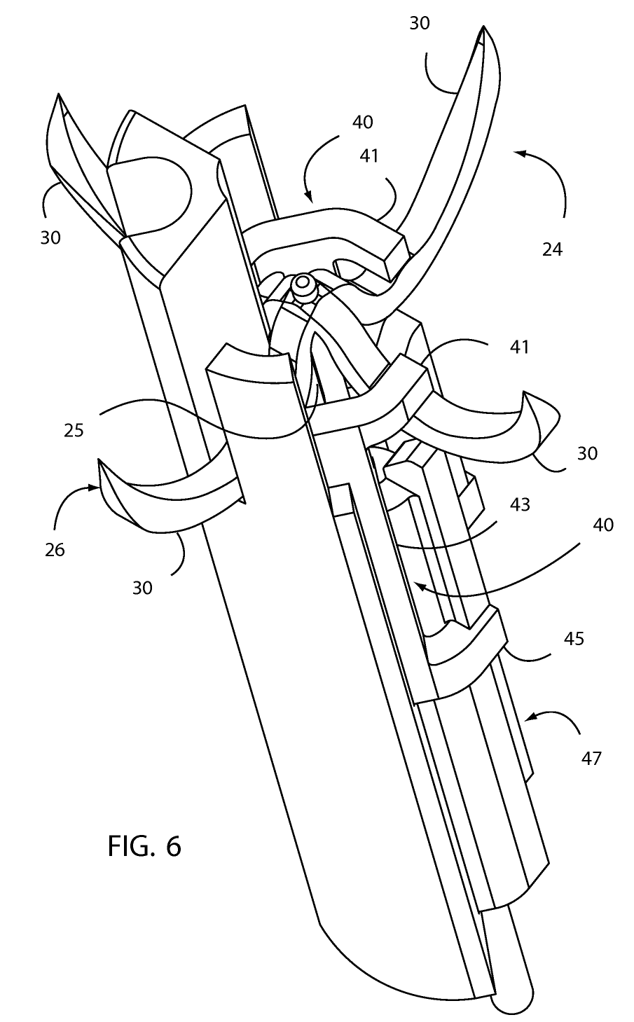

Any suitable mechanism or mechanisms may be utilized to splay and deploy the clasp 24 in the manner described in greater detail below. As one example, referring also to FIG. 7, at least one driver 38 may be located in the clasp applicator 16 proximal to the clasp 24, and may be movable relative to the clasp 24 such as by sliding. For clarity, only one clip 26 of the clasp 24 and part of the driver 38 is shown in FIG. 7. Advantageously, the driver 38 is symmetrical such that its interaction with each clip 26 is substantially the same. Alternately, the driver 38 need not be symmetrical, such that each clip 26 is splayed and deployed in a different manner, and/or at a different time in the actuation sequence. The clasp applicator 16 may include a passage defined therein that accommodates at least part of the driver 38 and guides the driver 38 during at least part of its motion. The driver 38 may be configured in any suitable manner. As one example, the driver 38 is elongated and has a bifurcated distal end, where each bifurcation is configured to engage a corresponding peak 32 of the clip 26. Alternately, the distal end of the driver 38 is shaped in any other suitable manner. Optionally, the clasp 24 may be fabricated integrally with the driver 38. If so, the clasp 24 is frangible or otherwise separable from the driver 38, such that the clasp 24 separates from the driver 38 at a suitable time during or after deployment. Alternately, the clasp 24 may be fabricated integrally with a holder 25, as shown in FIG. 6, and is frangible or otherwise separable from the holder 25 at a suitable time during or after deployment. The driver 38 may be connected to a cable, rod, or any other mechanism capable of moving the driver 38.

At least one splay arm 40 may be located in the clasp applicator 16. Each splay arm 40 may be located adjacent to a corresponding driver 38. Alternately, at least one splay arm 40 is positioned differently. Each splay arm 40 may be substantially planar, may be generally elongated in the longitudinal direction, and may be thin. Alternately, at least one splay arm 40 may be configured differently; for example, at least one splay arm 40 may be at least partially non-planar. Each splay arm 40 may be stamped from a thin sheet of metal or other material, or may be otherwise fabricated. Each splay arm 40 may be oriented relative to a corresponding driver 38 such that the longitudinal centerlines of the splay arm 40 and corresponding driver 38 are substantially parallel. Alternately, each splay arm 40 and corresponding driver 38 may be oriented differently relative to one another. Each splay arm 40 may be bifurcated to form two fingers 42 at the distal end thereof, each of which may be shaped in any suitable manner. Alternately, at least one splay arm 40 forms more than two fingers 42 at its distal end, or is not bifurcated or otherwise split into two or more fingers 42. At least one splay arm 40, and/or at least one finger 42, may be biased toward or away from a corresponding driver 38. Alternately, at least one splay arm 40 and/or finger 42 is not biased relative to a corresponding driver 38. A splay post 44 may extend from at least one finger 42 of a splay arm 40, or from a different part of the splay arm 40 than the finger 42. The splay post 44 is oriented toward the corresponding driver 38, and may be shaped or configured in any suitable manner. The splay post 44 may be a generally cylindrical protrusion, a bump, or any other suitable shape. Each splay post 44 is positioned such that it can splay a clip 26 of the clasp 24, as described in greater detail below. When the clasp applicator 16 is in the initial position, each splay post 44 may be in contact with a valley 34 of a clip 26 of the clasp 24. In this way, the splay posts 44 may act to restrain the clasp 24 within the clasp applicator 16 in the initial configuration, prior to deployment of the clasp 24. Alternately, at least one splay post 44 is positioned differently relative to the clasp 24 when the clasp applicator 16 is in the initial configuration.

One or more ears 46 may extend laterally, or in a different direction, from a splay arm 40 and/or finger 42. Each ear 46 may be configured to engage a ramp, aperture, or other feature defined in the housing 20. Interaction between each ear 46 and the housing 20 may move the associated splay arm 40 and/or finger 42 relative to the driver 38, as described in greater detail below. Alternately, one or more ears 46 may engage the driver 38 or other mechanism in the clasp applicator 16 to move the associated splay arm 40 and/or finger 42 relative to the driver 38. Alternately, the ears 46 are not used, and a different structure, mechanism or method is used to move the splay arm 40 and/or finger 42 relative to the driver 38.

Referring to FIG. 6, where the clasp 24 is generally X-shaped, the splay arms 40 may be configured to rotate out of the way of the clasp 24 after splaying it. At least one splay arm 40 may have a generally I-shaped, L-shaped or T-shaped configuration. At least one splay arm 40 may include a distal bar 41 configured to engage at least one tine 30, where the distal bar 41 may be spaced away from and at least partially curved or bent about the longitudinal centerline of the clasp 24. Such curvature advantageously allows one distal bar 41 to engage two or more tines 30 of the clasp 24. Alternately, at least one distal bar 41 may be shaped in a different manner. A longitudinal bar 43 may extend generally longitudinally in the proximal direction from a corresponding distal bar 41. The longitudinal bar 43 may extend from an end of the corresponding distal bar 41, or from a location between the ends of the corresponding distal bar 41. The proximal end of at least one longitudinal bar 43, or a different part of at least one longitudinal bar 43, may be connected to a proximal bar 45. At least one proximal bar 45 may be shaped in generally the same manner as the corresponding distal bar 41 of the splay arm 40. At least one proximal bar 45 may be shaped to slide along, and rotate about, a spindle 47. The surface of the spindle 47 may be spaced away from the longitudinal centerline of the clasp 24. As described in greater detail below, sliding of at least one splay arm 40 along the spindle 47 splays the clasp 24, and rotation of at least one splay arm 40 about the spindle 47 moves the distal bars 41 out of the path of the clasp.

Referring to FIG. 7, at least one anvil 48 initially may be positioned distal to the clasp 24. Advantageously, an anvil 48 is initially positioned distal to the trough 36 of each clip 26 of the clasp 24, such that each trough 36 contacts a corresponding anvil 48 during closing of that clip 26, as described below. The longitudinal centerline of each clip 26 may substantially intersect or approach close to intersection with the corresponding anvil 48. As a result, each peak 32 of each clip 26 may be offset from the anvil 48. At least one anvil 48 may be substantially fixed relative to the housing 20

At least one anvil 48 may be configured to move from its initial position distal to the corresponding clip 26 to a second position out of the path of the clasp 24 after the clasp 24 has been closed, as described in greater detail below. Any structure, mechanism or method may be used to move each anvil accordingly. As one example, referring also to FIG. 8, an anvil assembly 49 at least one anvil 48 may be positioned at or near the distal end of a rod 50 or similar elongated member, where at least the distal end of that rod 50 may be moved in one or more directions relative to the clasp 24. A collar 52 may encircle two or more rods 50 at a location proximal to the anvils 48. The proximal ends of the rods 50 may be located closer to one another than the distal ends of the rods 50. After closure of the clips 26 of the clasp 24, the anvils 48 may be moved out of plane with the clips 26 such as by moving the collar 52 distally, causing the distal ends of the rods 50 and thus the anvils 48 to move closer to one another. The rods 50 also may be moved in a different direction, such that the anvils 48 do not interfere with the backbone 28 of the clasp 24 as the clasp 24 exits the clasp applicator 18. Alternately, a single bifurcated rod 50 may be used, with an anvil 48 on each bifurcation. Alternately, the rods 50 are not used, and the anvils 48 are controlled in a different manner and/or with a different structure or mechanism; any mechanism that enables at least one anvil 48 to move out of the path of a corresponding clip 26 may be used.

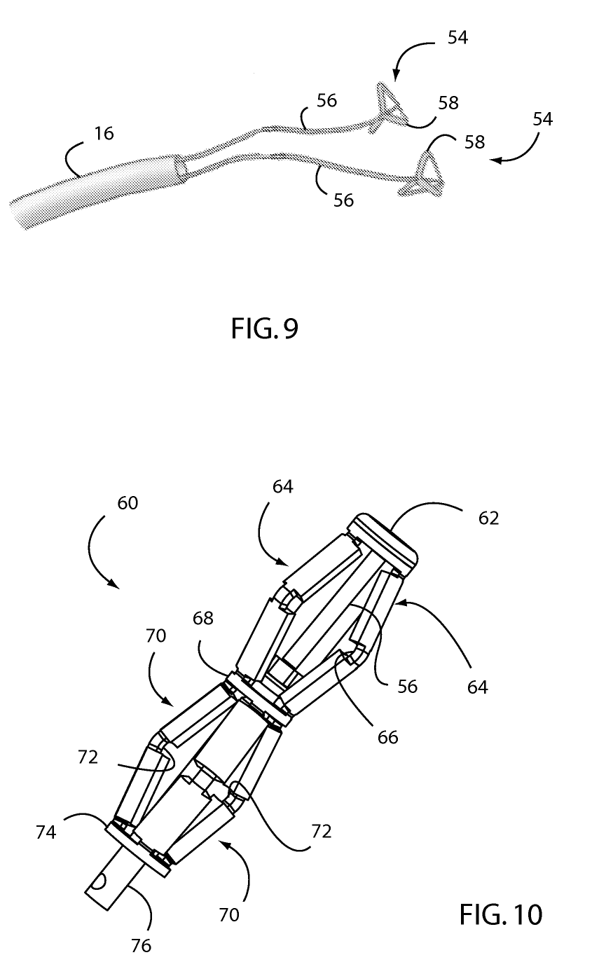

Referring to FIG. 9, at least one anchor 54 may be utilized in conjunction with the clasp applicator 16. Each anchor 54 is a mechanism with at least a portion thereof movable from a first configuration having a first width to a second configuration having a second, larger width, then back to the first configuration. Each anchor 54 may be configured in any suitable manner. As one example, at least one anchor 54 may be a tubular structure having two or more slots defined therethrough, such that compression of the anchor causes the segments 58 of the anchor 54 between the slots to deform outward. At least one anchor 54 may have a blunt or soft distal end. As one example, a soft tip fabricated from rubber, silicone or other soft biocompatible material may be connected to the distal end of at least one anchor 54. As another example, one or more anchors 54 may be dipped in a material that solidifies into a soft surface for bluntness. As another example, one or more anchors may be coated in a soft material for bluntness, in any suitable manner.

Each anchor 54 may be connected to a corresponding guidewire 56. Where the corresponding anchor 54 is originally in a substantially tubular state and/or is configured to have a lumen therethrough, the guidewire 56 may extend into that lumen and connect to the distal end of the anchor 54. The guidewire 56 may be tensioned in order to compress the corresponding anchor 54 and cause segments 58 thereof to expand outward. At least one anchor 54 may be elastically deformable. If so, the anchor 54 is in the first configuration until the guidewire 56 is tensioned to deform the anchor 54 to the second configuration, and the anchor 54 remains in the second configuration until the guidewire 56 is released Alternately, at least one anchor 54 may be superelastically deformable. For example, such an anchor 54 may be fabricated from nickel-titanium alloy. Alternately, at least one anchor 54 may be plastically deformable. Such an anchor 54 may be substantially as disclosed in U.S. patent application Ser. No. 11/093,003. The housing 20 of the clasp applicator 16 may include at least two apertures defined therein, where at least one guidewire 56 passes through an aperture different from at least one other guidewire 56. The passage of guidewires 56 through different apertures allows the clasp applicator 16 to be properly located relative to the PFO 2, as described in greater detail below. Alternately, at least one anchor may is connected instead, or in addition, to a structure other than a guidewire.

The housing 20 may be configured for rapid exchange along at least one guidewire 56 and/or a different guidewire placed separately from the PFO closure system 12. That is, the housing 20 may include at least one aperture defined in a side thereof (not shown) associated with a corresponding guidewire 56, where that guidewire 56 extends into the housing 20 through the aperture; proximal to that aperture, the guidewire 56 is located beside the catheter 14. A corresponding second aperture may be defined in a side of the housing 20, distal to the first aperture, through which the guidewire 56 extends distally out of the housing 20. In this way, only a short length of the overall guidewire 56 is located within the housing 20 at any given time. Alternately, the housing 20 may be configured to allow over-the-wire usage with respect to at least one guidewire 56, where that guidewire 56 extends into an opening or aperture in the proximal end of the housing 20 and may also extend out of an opening or aperture in the distal end of the housing 20.

Referring to FIG. 10, as another example of an anchor 54, at least one anchor 54 may be a basket 60 attached to a guidewire 56. The basket 60 may include a base 62, which is connected to the distal end of the guidewire 56. Advantageously, the guidewire 56 does not extend distal to the base 62. The base 62 is configured to be atraumatic; for example, the base 62 may be blunt. Alternately, an atraumatic tip (not shown) may extend distally from the base 62. Alternately, the base 62 is part of the guidewire 56, rather than the basket 60. Alternately, a different part of the basket 60 is connected to the guidewire 56. Alternately, the basket 60 may be connected to a different part of the guidewire 56 than the distal end thereof. At least one distal strut 64 is connected to the base 62, where each distal strut 64 extends in a generally proximal direction from the base 62. Each distal strut 64 may form a shallow V-shape in its initial configuration, where the point of the V-shape is oriented outward from the longitudinal centerline of the basket 60. Alternately, at least one distal strut 64 may be shaped in a different manner. Each distal strut 64 includes a notch 66 defined therein substantially at the point of the V-shape of that distal strut 64. Each notch 66 may be shaped in any suitable manner, and may be made in any surface of the corresponding distal strut 64. The proximal end of each distal strut 64 is connected to a ring 68, where the guidewire 56 passes through the opening in the ring 68. Alternately, any other suitable structure may be utilized instead of the ring 68, such as a plate having an aperture therein, a structure having an open perimeter, or one or more bars. Alternately, the ring 68 may be omitted altogether.

At least one proximal strut 70 extends proximally from the ring 68. Where the ring 68 is omitted, each proximal strut 70 is connected directly to the corresponding distal strut 64. Each proximal strut 70 may form a shallow V-shape in its initial configuration, where the point of the V-shape is oriented outward from the longitudinal centerline of the basket 60. Alternately, at least one proximal strut 70 may be shaped in a different manner. Each proximal strut 70 includes a notch 72 defined therein substantially at the point of the V-shape of that proximal strut 70. Each notch 72 may be shaped in any suitable manner, and may be made in any surface of the corresponding proximal strut 70. Each notch 72 in a proximal strut 70, and each notch 66 in a distal strut 64, is defined therein in such a manner that when a compressive force is applied to the basket 60, the distal struts 64 bend before the proximal struts 70. As one example, the notches 66 in the distal struts 64 may be deeper and/or larger than the notches 72 in the proximal struts 70. Alternately, the notches 66, 72 are defined in the corresponding struts 64, 70 such that the proximal struts 70 bend before the distal struts 64, or such that the struts 64, 70 bend substantially at the same time. The proximal end of each proximal strut 70 is connected to an endcap 74 at the proximal end of the basket 60. Alternately, a different part of each proximal strut is connected to the endcap 74. Alternately, the proximal struts 70 are connected directly to the guidewire 56. Alternately, a different structure than the endcap 74 is used. Alternately, the endcap 74 may be omitted.

The guidewire 56 may be tensioned in order to compress the basket 60 connected thereto, and cause the struts 64, 70 to expand outward. Such tension exerts a compressive force on the basket 60. Due to the shallow V-shape of the struts 64, 70, that compressive force results in the ends of each V-shape moving closer to one another, expanding the struts 64, 70 outward. Where the notch 66 in each distal strut 64 is deeper, larger or otherwise configured differently from the notch 72 in each proximal strut 70, the compressive force on the basket 60 causes the distal struts 64 to expand before the proximal struts 70. That is, the energy of compression first deforms the distal struts 64, which offer less resistance to that energy, then deforms the proximal struts 70 after deformation of the distal struts 64 is complete. Alternately, the notches 66, 72 are configured such that the struts 64, 70 deform in the opposite order, or at substantially the same time. After the struts 64, 70 have been deformed outward, the basket 60 is in the second configuration.

The struts 64, 70 may be elastically deformable, superelastically deformable, or plastically deformable. Alternately, at least one strut 64, 70 is deformable in one manner, and at least one other strut 64, 70 is deformable in a different manner. Where the struts 64, 70 are elastically or superelastically deformable, the basket 60 is in the first configuration as shown in FIG. 10 until the guidewire 56 is tensioned to deform the basket 60 to the second configuration, and the basket 60 remains in the second configuration until the guidewire 56 is released. Where the struts 64, 70 are plastically deformable, the basket 60 is in the first configuration as shown in FIG. 10 until the guidewire 56 is tensioned to deform the basket 60 to the second configuration, and the basket 60 remains in the second configuration until a tensile force is exerted on it. The guidewire 56 connected to each basket 60 may pass through a jacket 76 having a lumen therein, where the lumen has a diameter at least as large as the diameter of the guidewire 56. The guidewire 56 is slidable within the lumen of the jacket 76. The jacket 76 may be both thin-walled and stiff enough to provide a reaction force against the compressive force exerted by the guidewire 56 against the basket 60. That is, the distal end of the jacket 76 may be connected to or positioned against the endcap 74 of the basket 60, such that tensioning the guidewire 56 compresses the basket 60 against the jacket 76 rather than simply moving the basket 60 proximally. Alternately, a different structure than the jacket 76 is utilized to provide a reaction force. As one example, the catheter 14 and/or the clasp applicator 16 provide a reaction force against which each basket 60 is compressed. Alternately, the basket 60 and the guidewire 56 are connected and/or configured such that no reaction force is necessary.

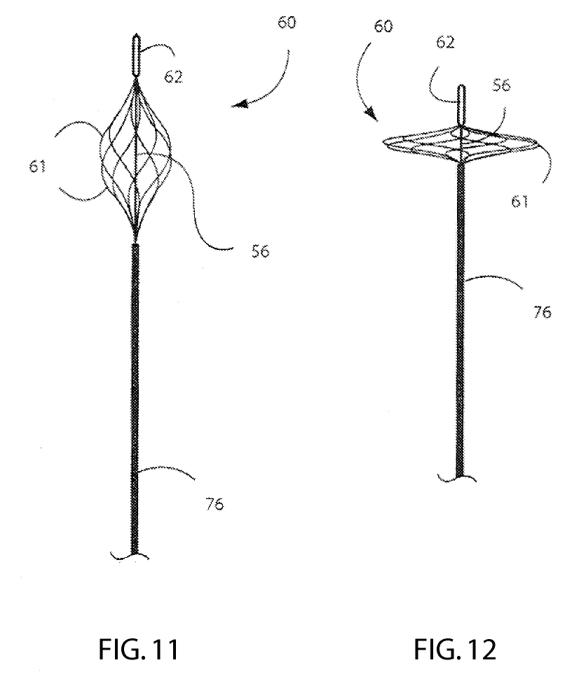

Referring to FIG. 11, another exemplary embodiment of the anchor 54 is shown. As in the previous embodiment, a guidewire 56 may extend generally longitudinally through the anchor 54. The distal end of the guidewire 56 may be attached to a base 62, which is atraumatic. In this way, the distal end of the guidewire 56 advantageously does not extend distal to the base 62. The base 62 may be configured in any suitable manner that allows it to engage tissue atraumatically. Alternately, the guidewire 56 may be omitted. In its initial position, the anchor 54 of FIG. 11 extends distally outward from the jacket 76, where the anchor 54 has a first diameter. Alternately, in the initial position the anchor 54 is collapsed to a diameter small enough to allow it to be held partially or completely within the lumen of the jacket 76. Alternately, the jacket 76 may be omitted. The anchor 54 may be formed from two or more wires 61 twisted about the guidewire 56. The distal ends of the wires 61 may be fixed to the base 62, but not the guidewire 56. Alternately, at least one wire 61 may be fixed to the guidewire 56. The proximal ends of the wires 61 may be connected to the distal end of the jacket 76, or may extend into the lumen of the jacket 76 any suitable length. Referring to FIG. 12, the anchor 54 may be expandable as a result of retraction of the base 62 relative to the jacket 76, when the guidewire 56 is pulled proximally and the jacket 76 is held in a generally constant position. After expansion, the anchor 54 is in an expanded position, and has a second diameter larger than the first diameter. The wires 61 may be made of any suitable material, and may be elastically deformable, superelastically deformable or plastically deformable.

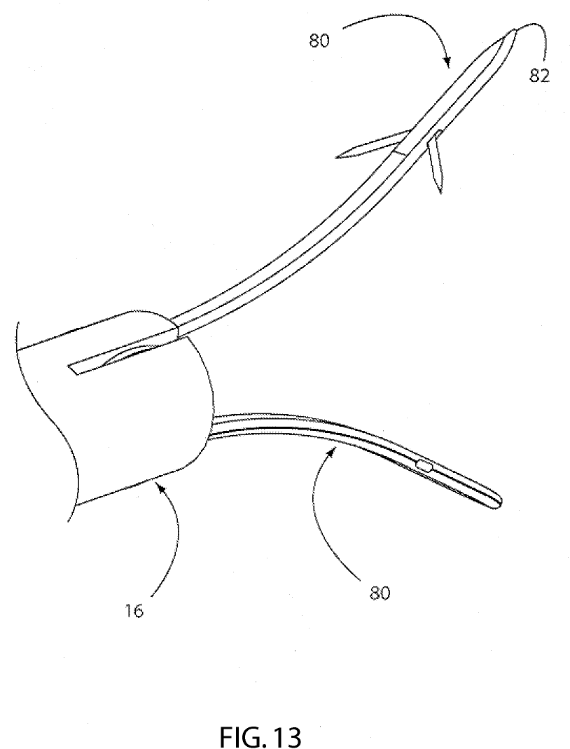

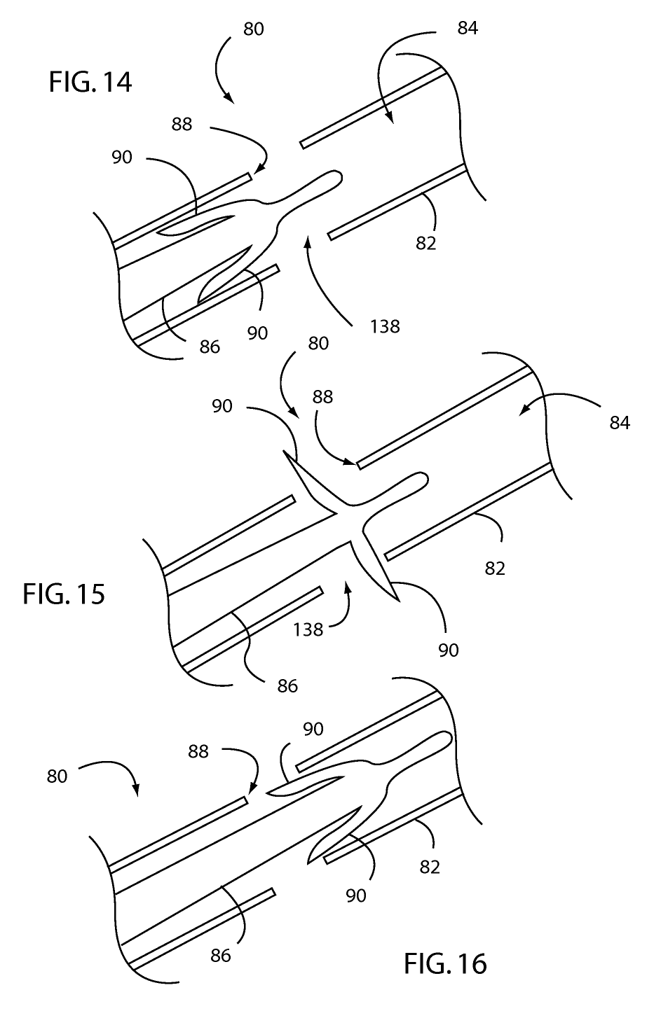

Referring to FIG. 13, at least one anchor 54 may be a harpoon 80. At least one harpoon 80 may be connected to the clasp applicator 16. At least one harpoon 80 may be additionally, or instead, connected to a corresponding guidewire 56. Each harpoon 80 has a sharp distal end 82 configured to penetrate tissue. Referring also to FIG. 14, each harpoon 80 may be substantially tubular along at least part of its length, where the tubular portion includes a tube wall 82 and a lumen 84 defined therein. The harpoon 80 may include a control wire 86 that extends into the lumen 84 of the harpoon 80. The control wire 86 may be part of at least one guidewire 56, may be a separate wire that extends into and is controlled by the clasp applicator 16, or may be any suitable structure other than a wire. As one example, a guidewire 56 may extend from the handle 18 of the PFO closure system 12 to the clasp applicator 16, where that guidewire 56 is operatively connected to one or more control wires 86, such that actuation of the guidewire 56 by the handle 18 actuates multiple control wires 86. The tube wall 82 includes at least one aperture 88 defined therein. At least one barb 90 may extend from the control wire 86, and may be biased outward from the control wire 86 toward the tube wall 82. Contact between each barb 90 and the inner surface of the tube wall 82 may deflect each barb 90 inward. This configuration of the harpoon 80 may be referred to as the initial configuration. Referring also to FIG. 15, as the control wire 86 is advanced distally within the harpoon 80, each barb 90 encounters a corresponding aperture 88, which is sized to allow the barb 90 to move outward from the harpoon 80 under its bias and extend a distance outward from the outer surface of the harpoon 80. The tip of each barb 90 is sharp, and extends at least partially in the proximal direction. In this way, each barb 90 resists proximal motion relative to tissue, as described in greater detail below. This configuration of the harpoon 80 may be referred to as the deployed or engaged configuration. Referring also to FIG. 16, as the control wire 86 is advanced further distally within the harpoon 80, engagement between each barb 90 and the distal edge of each aperture 88 pushes each barb 90 back toward the control wire 86 and then into the lumen 84 of the harpoon 80. After the control wire 86 advances distally far enough, each barb 90 is positioned within the lumen 84 of the harpoon 80, distal to the corresponding aperture 88. Contact between each barb 90 and the inner surface of the tube wall 82 may deflect each barb 90 inward. This configuration of the harpoon 80 may be referred to as the withdrawal configuration of the harpoon 80. Multiple apertures 88 may be present in the harpoon 80, and may be located at substantially the same longitudinal position as one another, or at different longitudinal positions from one another. More than one barb 90 may extend from a single aperture 88. Alternately, the control wire 86 may be retracted proximally rather than advanced distally, such that the barb 90 is actuated in substantially the opposite sequence as described above.

Alternately, one or more anchors 54 may be omitted, and one or more different structures, mechanisms or methods are instead used to anchor the clasp applicator 16 in place relative to the PFO 2. As one example, suction may be used to hold the clasp applicator 16 and/or the catheter 14 in place against tissue of the heart 10 in proximity to the PFO 2. A vacuum is applied to the clasp applicator 16 and/or catheter 14, and one or more apertures (not shown) in the clasp applicator 16 and/or catheter 14 transmit that vacuum to the tissue of the heart 10 in proximity to the PFO 2, generating suction. Advantageously, such suction is not applied to the flaps of the PFO 2. Such suction may be applied to tissue of the heart 10 in proximity to, or at a location spaced apart from, the PFO 2. Alternately, such suction may be used in addition to, rather than instead of, at least one anchor 54.

Operation

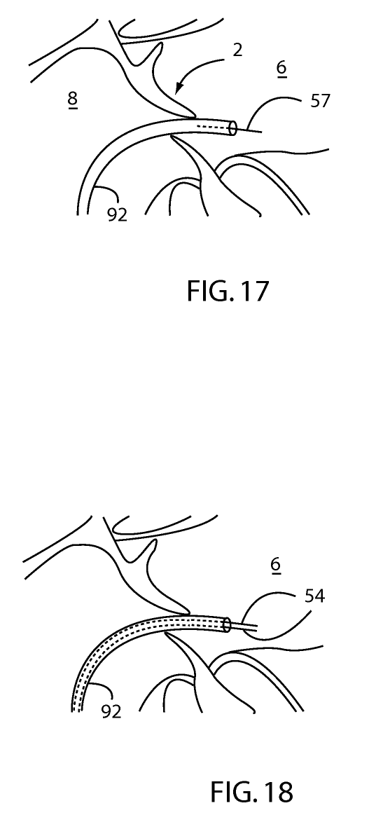

An exemplary method of placing the PFO closure system 12 in a patient and actuating the PFO closure system 12 is described here. However, the PFO closure system 12 may be placed in a patient and/or actuated in any other suitable manner, such as by the use of a guidewire without a catheter, or a catheter without a guidewire. Alternately, neither a catheter nor a guidewire is used to place the PFO closure system 12 in position in a patient. In one example of placement of the PFO closure system 12 in a patient, initially, the femoral artery, radial artery or other artery or vein in the vasculature remote from the PFO 2 is punctured, and a standard introducer sheath is placed into the puncture. Such access to the vasculature is standard. Referring to FIG. 17, a guidewire 57 of the user's choosing is inserted through the introducer sheath. Optionally, the guidewire 57 may include a standard radiopaque feature at or near its distal end, to aid positioning of the distal end of the guiding catheter 92 relative to the PFO 2. The guidewire 56 is advanced through the vasculature to the PFO 2, then completely through the PFO 2 into the left atrium 6. A guiding catheter 92 is then inserted through the introducer sheath over the guidewire 56 and advanced through the vasculature to the PFO 2. The distal end of the guiding catheter 92 is then advanced completely through the PFO 2 into the left atrium 6. Alternately, the guiding catheter 92 and the guidewire 57 may be inserted substantially simultaneously, in which case the guidewire 57 may be located completely within a lumen of the guiding catheter 92, or may extend distally from the end of the guiding catheter 92, during this advancement. Advancement of the guiding catheter 92 and at least one guidewire 57 advantageously may be performed with the assistance of a fluoroscope or other imaging device that indicates the position of the guiding catheter 92 and/or at least one guidewire 57 in the patient. The use of such an imaging device in conjunction with a guiding catheter 92 and/or at least one guidewire 57 is standard in the art. The guidewire 57 may then be removed from the guiding catheter 92. Alternately, a guidewire 56 connected to an anchor 54 is utilized, in which case that guidewire 56 may be left in place in the guiding catheter 92.

The distal end of the PFO closure system 12 is then advanced through the lumen of the guiding catheter 92 into the left atrium 6. Referring also to FIG. 18, at the end of this advancement, the distal ends of the anchors 54 may extend out of the distal end of the guiding catheter 92. At this time, each anchor 54 advantageously is in the first configuration.

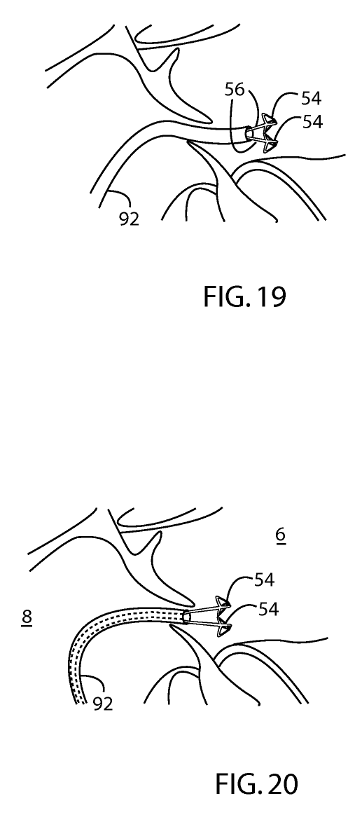

Referring also to FIGS. 9 and 19, the anchor 54 associated with each guidewire 56 is actuated to expand outward to the second configuration, in which the anchor 54 has a width greater than it had in the first configuration. Referring also to FIG. 10, where at least one of the anchors 54 is a basket 60, the guidewire 56 attached to that basket 60 is retracted proximally to cause the distal struts 64 to deform outward, expanding the diameter of the basket 60. That is, the guidewire 56 exerts a compressive force on the basket 60, where that compressive force is great enough to cause deformation of the distal struts 64, but not great enough to cause deformation of the proximal struts 70. Alternately, the proximal struts 70 are configured to expand along with the distal struts 64 upon the application of the same amount of force. Where the basket 60 is configured as shown in FIGS. 11-12, the guidewire 56 is retracted proximally, such that the base 62 is retracted toward the jacket 76, which is held in a generally constant position. This relative motion of the base 62 and the distal end of the jacket 76 compresses the wires 61 of the basket 60, causing it to expand radially from its first diameter to its second diameter. Where suction is used to hold the PFO closure catheter 14 against tissue in proximity to the PFO 2, such suction may be applied now, or may be applied at any suitable time during the procedure, either before or after this time, or both.

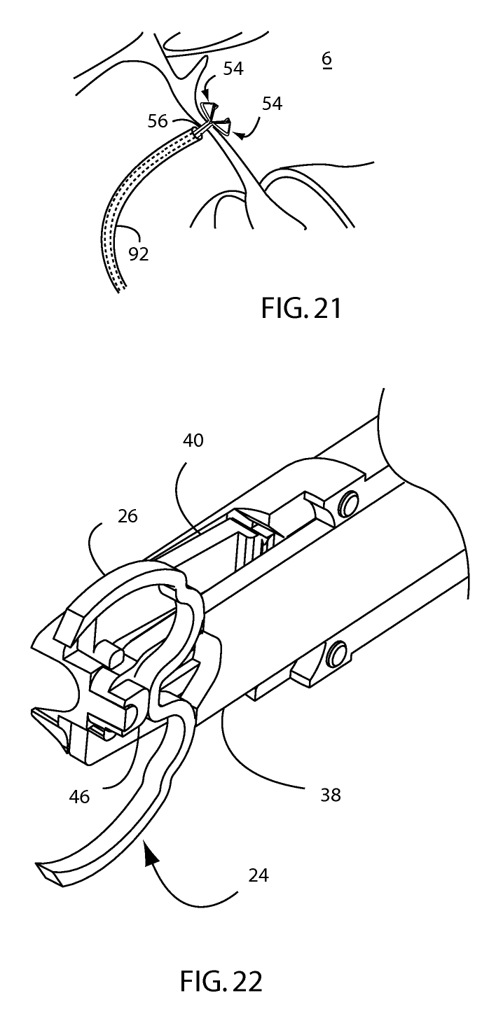

Next, referring also to FIG. 20, the PFO closure catheter 14 and guidewires 56 are retracted proximally. The distal end of the PFO closure catheter 14 moves out of the PFO 2 and into the right atrium 8. Referring also to FIG. 21, the anchors 54, with their distal struts 64 expanded, encounter the left atrial side of the PFO 2, pushing the flaps of the PFO 2 into overlapping contact with each other, or bringing the flaps into closer proximity with one another. As the anchors 54 move toward the PFO 2, they engage heart tissue adjacent to the PFO 2. In the expanded state, the anchors 54 are too large to pass through the PFO 2. Thus, when the anchors 54 move proximally, they engage the flaps of the PFO 2 and may also engage heart tissue near the PFO 2, and do not pass through the PFO 2. Such engagement between the anchors 54 and tissue may be referred to as “anchoring.” At this point, the distal end of the PFO closure catheter 14 is spaced apart from the PFO 2. Where at least one anchor 54 is a basket 60, the proximal struts 70 may then be expanded outward, such that the proximal struts 70 engage tissue on one side of the PFO 2 and the distal struts 64 engage tissue on the other side of the PFO 2.

Referring also to FIG. 13, where at least one anchor 54 is a harpoon 80, anchoring of the PFO closure catheter 14 to the PFO 2 may be performed differently. As described above, the guiding catheter 92 is moved to a position in the right atrium 8 adjacent to the PFO 2 but does not cross the PFO 2 into the left atrium 6. Instead, referring also to FIG. 13, the clasp applicator 16 at the distal end of the PFO closure catheter 14 is placed in proximity to the PFO 2, and then one or more harpoons 80 are advanced distally from the clasp applicator 16 to engage the flaps of the PFO 2 or heart tissue in proximity to the PFO 2. The harpoons 80 may be particularly useful in closing a PFO 2 that is more tunnel-like. The harpoons 80 may be curved or otherwise configured to move outward from the longitudinal centerline of the clasp applicator 16 as they are advanced distally, in order to capture heart tissue on either side of the PFO 2. As the harpoons 80 are advanced into heart tissue, they may be configured as shown in FIG. 14.

Referring also to FIG. 15, the control wire 86 then is advanced distally within the harpoon 80, causing each barb 90 to encounter a corresponding aperture 88 in the tube wall 82 of the harpoon 80. Each barb 90 then moves outward from the harpoon 80 under its bias, and extends outward from the outer surface of the harpoon 80. The tip of each barb 90 is sharp, and extends at least partially in the proximal direction. This orientation of the barbs 90 prevents the harpoon 80 from moving proximally, by engaging heart tissue in a way that sets the barbs 90 more securely if the harpoons attempt to move proximally.

Where the PFO 2 is a tunnel-like PFO 2, the PFO closure catheter 14 may be advanced into the PFO 2 such that the clasp applicator 16 is located within the PFO 2, and is then left in the PFO 2 rather than retracted proximally. The anchors 54 then may be retracted relative to the clasp applicator 16 to affirmatively engage the barbs 90 with heart tissue and thereby hold the clasp applicator 16 in place in the PFO 2.

For clarity and simplicity, splaying and closing of the multiple clips 26 of a clasp 24 is described and illustrated with regard to one clip 26; the other clips 26 are splayed and closed in the same or similar manner. Alternately, different clips 26 may be splayed and/or closed differently, according to different methods described herein. With the clasp applicator 16 anchored to the PFO 2, the clasp applicator 16 is then actuated to splay the clasp 24. To do so, at least one of the splay arm 40 and the driver 38 may move relative to the other. As one example, the driver 38 is held substantially stationary, and the splay arm 40 is moved proximally. The splay arm 40 may be actuated to retract proximally in any suitable manner. As one example, the splay arm 40 is urged proximally when the handle 18 exerts a proximal force on a cable or other structure or mechanism connected to the splay arm 40. However, the driver 38 may be actuated in any other suitable manner.

Referring to FIGS. 7 and 22, as the splay arm 40 retracts proximally, each splay post 44 exerts a proximal force on the corresponding valley 34 of each clip 26 of the clasp 24. The driver 38 corresponding to that clip 26 remains substantially in the same position as the splay arm 40 retracts proximally, and thereby substantially restrains the trough 36 of that clip 26 against proximal motion, such that the longitudinal position of the trough 36 is substantially unchanged as the splay posts 44 exert proximal force on the valleys 34 of the clip 26. However, the tines 30 of the clip 26 are not substantially restrained against motion resulting from application of force to the clip 26 by the splay posts 44. Each splay post 44 is positioned sufficiently far from the longitudinal centerline of the clip 26 such that the exertion of proximal force by that splay post 44 against a corresponding valley 34 of the clip 26 (which itself is spaced apart from the longitudinal centerline of the clip 26 generates a moment about the trough 36 of the clip 26. This moment causes the corresponding tine 30 of the clip 26 to move outward from the longitudinal centerline of the clip 26. Thus, as the splay posts 44 exert a force on the corresponding clip 26, the distal ends of the tines 30 of the clip 26 each move in a direction having a component of motion away from the longitudinal centerline of the clip 26. This deformation of the clip 26 may be referred to as “splaying.” During splaying of the clip 26, the tines 30 themselves may remain substantially undeformed; rather, a portion of the clip 26 in proximity to each valley 34 and/or trough 36 may deform. Alternately, at least one tine 30 may deform during splaying of the clip 26. Optionally, where at least one tine 30 of at least one clip 26 includes a splay bump 29 defined on its inner surface, at least one splay post 44 may engage a corresponding splay bump 29. Exertion of proximal force by at least one splay post 44 against a corresponding splay bump 29 causes the corresponding tine 30 to splay outward, after which the splay post 44 may move into the corresponding valley 34 of the clip 26 in order to continue splaying substantially as described earlier in this paragraph.

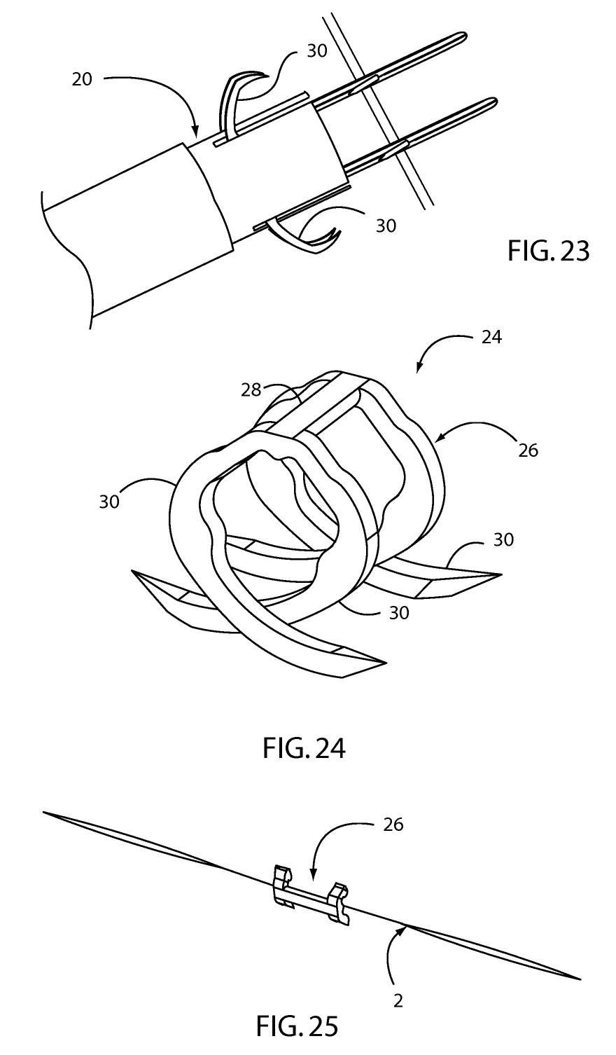

Referring also to FIG. 23, as the distal ends of the tines 30 move away from the longitudinal centerline of the clip 26 during splaying of the clip 26, at least part of each tine 30 may move outside the distal end of the housing 20 of the clasp applicator 16 through a slot 22 or other opening in the housing 20. As a result, the tines 30 of the clip 26 may move apart from one another a distance greater than the diameter of the housing 20. Where the clip 26 is made from a plastically-deformable material such as stainless steel, the clip 26 deforms plastically as it splays from its initial configuration to the splayed configuration. Plastic deformation is deformation that remains after the load that caused it is removed, or that would remain if the load were removed. Alternately, at least one clip 26 is elastically deformable from its initial configuration to the splayed configuration. At least one elastically-deformable clip 26 may be spring-loaded inwards to the initial configuration, such that the clip 26 springs outward and returns to the splayed configuration upon application of force or upon movement to a position relative to the housing 20 such that the clip 26 is free to spring outward.

As another example, the splay arm 40 may be held substantially stationary while the driver 38 may be actuated to advance distally to splay the clasp 24. The driver 38 may be actuated to advance distally in any suitable manner. As one example, the driver 38 is urged distally when the handle 18 exerts a proximal force on a cable or other structure or mechanism connected to the driver 38. Such a cable may have a pathway within the clasp applicator 16 such that proximal motion of the cable results in distal motion of the driver 38. For example, the cable may be connected to the driver 38, and that cable may be directed around a nose, axle or other feature (not shown) located distal to the driver 38 in order to convert proximal motion of the cable to distal motion of the driver 38. However, the driver 38 may be actuated in any other suitable manner. As the driver 38 advances distally, it exerts a distal force on the trough 36 of the corresponding clip 26. The splay posts 44 corresponding to that clip 26 substantially restrain the valleys 34 of that clip 26 against distal motion, such that the longitudinal position of the valleys 34 is substantially unchanged as the driver 38 exerts distal force on the trough 36 of the clip 26. However, the tines 30 of the clip 26 are not substantially restrained against motion resulting from application of force to the clip 26 by the driver 38. Each splay post 44 is positioned sufficiently far from the longitudinal centerline of the clip 26 such that the exertion of distal force by the driver 38 against the trough 36 substantially along that longitudinal centerline generates a moment about each splay post 44, and thus about each valley 34 of the clip 26. This moment causes the corresponding tine 30 of the clip 26 to move outward from the longitudinal centerline of the clip 26, splaying the clip 26. As described above, during splaying the distal ends of the tines 30 may move outside the distal end of the housing 20 of the clasp applicator 16 through a slot 22 or other opening in the housing 20. Advantageously, as described above, each clip 26 deforms plastically as it splays from its initial configuration to the splayed configuration.

As another example, the splay arm 40 is moved proximally and the driver 38 is moved distally at generally the same time, to splay the corresponding clip 26 in substantially the same manner as described above. Alternately, at least one of the clips 26 may be splayed in any other suitable manner.

The clasp applicator 16 optionally may splay the clasp 24 beyond ninety degrees. That is, the clasp 24 may be moved such that a line formed by the distal end of one of its tines 30 and the most distal point on the trough 36 forms an angle of greater than ninety degrees with the longitudinal centerline of the clasp 24. Splaying the clasp 24 beyond ninety degrees may be advantageous in closing a tunnel-like PFO 2 or other PFO 2.

Splaying of the clasp 24 occurs in substantially the same manner as described with regard to the clasp 24 configured as in FIG. 4. As set forth above, “splaying” refers to the motion of the distal ends of the tines 30 each in a direction having a component of motion away from the longitudinal centerline of the clasp 24.

Next, the distal end of the clasp applicator 16 is moved into contact with the PFO 2. Such motion may be accomplished simply by pushing the clasp applicator 16 distally along the lumen of the guiding catheter 92, or may be accomplished in any other suitable manner. At this point, the distal end of the clasp applicator 16 is substantially aligned with the PFO 2, and is adjacent to the PFO 2. The clasp applicator 16 may be characterized as being registered to the PFO 2 in this position. The clasp applicator 16 is also held substantially in place relative to the PFO 2 at this time. The location of the distal end of the clasp applicator 16 may be determined with a fluoroscope or other imaging device, and/or may be determined physically, by holding the PFO 2 on the right atrial side with the anchors 54 while simultaneously advancing the PFO closure catheter 14 until it encounters resistance. Where the anchors 54 are harpoons 80, the clasp applicator 16 may be actuated to retract the harpoons 80. Such actuation causes the distal end of the clasp applicator 16 to move against or in proximity to the PFO 2, because the barbs 90 of the harpoon 80 prevent it from moving proximally. In this way, the clasp applicator 16 is registered to the PFO 2.

Referring also to FIG. 23, each clip 26 is in the splayed configuration as the clasp applicator 16 moves into registration with the PFO 2. As a result, the distal ends of the tines 30 penetrate tissue in the vicinity of the PFO 2, and/or the flaps of the PFO 2 itself, as the clasp applicator 16 moves into registration with the PFO 2. The distal ends of the tines 30 of a clip 26 are positioned further apart from one another when the clip 26 is in the splayed configuration than when the clip 26 is in the initial configuration, thereby allowing capture of tissue across a width greater than that of the housing 20 of the clasp applicator 16 between the tines 30 as they enter and penetrate tissue. Alternately, at least one clip 26 is not splayed until the clasp applicator 16 is registered to the PFO 2.

Next, the clasp 24 is deformed to a closed configuration. Prior to this deformation, or during this deformation, the splay arm or arms 40 are moved away from the clasp 24 to allow the tines 30 of the clasp 24 to deform to the closed configuration. Where the splay arm 40 is configured as shown in FIG. 7, the driver 38 and/or splay arm 40 may be shaped such that contact between them as the driver 38 moves distally may push the splay arm 40 and the splay posts 44 away from the longitudinal centerline of the clasp 24 and thus out of the way of the clasp 24. Where the splay arm or arms 40 are configured as shown in FIG. 6, the splay arm or arms 40 may be rotated about the spindle 47 to move the distal bars 41 out of the way of the clasp 24. As another example, where one or more ears 46 extend from a splay arm 40, each ear 46 may engage a ramp, aperture, or other feature defined in the housing 20, such that interaction between each ear 46 and the housing 20 moves the associated splay arm 40 away from the longitudinal centerline of the clasp 24 and thus out of the way of the clasp 24. Alternately, any suitable structure, mechanism or method may be used to move the splay arm or arms 40 out of the way of the clasp 24 before the clasp 24 is closed.

As one example of closing the clasp 24, the driver 38 may be actuated to apply a distal force to each peak 32 of each clip 26. Each peak 32 of a clip 26 is offset from the longitudinal centerline of the clip 26. Further, the longitudinal centerline of the clip 26 substantially intersects or approaches close to intersection with the anvil 48. As a result, each peak 32 of a clip 26 is laterally offset from the corresponding anvil 48. The trough 36 is in contact with a corresponding anvil 48. Alternately, as at least one driver 38 moves distally, it moves a corresponding clip 26 such that its trough 36 engages a corresponding anvil 48. The force exerted by the driver 38 distally on each peak 32 of a clip 26 thus results in a moment about the anvil 48. Each tine 30 of a clip 26 consequently experiences that moment and moves toward the longitudinal centerline of the clip 26, deforming the clip 26. This deformation of the clip 26 may be plastic deformation from the splayed configuration to a final, closed configuration. In the course of this motion, referring also to FIG. 24, the distal ends of the tines 30 may first move toward the longitudinal centerline of the clip 26 and toward one another, swipe past each other, then move away from the longitudinal centerline of the clip 26 and away from one another. Thus, when the clip 26 is closed, the tines 30 may be both offset from and substantially adjacent to one another. The tines 30 need not substantially change shape as they move; rather, they may rotate about a pivot point located at or near the trough 36. Alternately, one or both of the tines 30 of each clip 26 may deform as they move. The radius of curvature of each tine 152 may be substantially coincident with its path of travel during closure of the clip 26.

The clasp 24 may be configured in any manner that allows the tines 30 to swipe past one another. The distal ends of the tines 30 of a clip 26 may be shaped substantially conically. As the clip 26 closes, the conical tips of the tines 30 come into contact with one another. As a result of the angle of the side of each conical tip, this contact causes the tines 30 to slide adjacent to one another instead of interfering with one another. Alternately, the distal end of each tine 30 may be substantially planar, where each plane is oriented in a different direction. As a result, when the distal ends of the tines 30 encounter one another, contact between the differently-oriented planes at the distal ends of the tines 30 pushes the tines 30 out of plane relative to one another. Alternately, the tines 30 of the clip 26 are fabricated such that they are out of plane with one another when the clip 26 is in the initial configuration, such that the tines 30 do not substantially interfere with one another during deployment. Alternately, the clip 26, driver 38 and/or splay arm 40 are configured to prevent the tines 30 from interfering with one another as the clip 26 closes. Alternately, at least two tines 30 of the clip 26 are configured to interfere with or otherwise engage one another when the clip 26 is in the closed position. Alternately, at least two tines 30 may be substantially parallel to one another and spaced apart from one another when the clip 26 is in the closed position. When deformation of the tines 30 of the staple is complete, each clip 26 is in the closed configuration, and the clasp 24 may be said to be in the closed configuration. In that closed configuration, at least part of each tine 30 advantageously engages the PFO 2 and/or tissue in proximity to the PFO 2, thereby holding the flaps of the PFO 2 and/or opposite sides of the of the PFO 2 together, closing the PFO 2.

Where the clasp 24 is configured as in FIGS. 5-6, closing of that clasp 24 occurs in substantially the same manner as described with regard to the clasp 24 configured as in FIG. 4. That is, the distal ends of the tines 30 may first move toward the longitudinal centerline of the clasp 24 and toward one another, swipe past each other, then move away from the longitudinal centerline of the clasp 24 and away from one another. Where the clasp 24 is frangibly connected to a holder 25, the clasp 24 may be separated from the holder 25 after closing. Alternately, the clasp 24 may be separated from the holder 25 during or before closing.

Next, the user may test the clasp 24 to ensure that the clasp 24 is firmly connected to tissue. Any suitable method may be used to ensure that the closed clasp 24 is firmly attached to the PFO 2 and/or tissue in vicinity of the PFO 2. As one example, the handle 18 is gently retracted in the proximal direction, thereby pulling on the PFO closure catheter 14 and the clasp applicator 16. If resistance is felt, then the clasp 24 is securely connected to tissue. If little or no resistance is felt, the PFO closure catheter 14 is removed from the patient, and along with it the closed clasp 24 held by the clasp applicator 16 at the distal end of the PFO closure catheter 14.

Next, if the clasp 24 is firmly connected to tissue, the clasp 24 is released from the clasp applicator 16. The clasp 24 may be passively released from the clasp applicator 16, such that one or more structures or mechanisms holding the clasp 24 in the clasp applicator 16 are moved or otherwise actuated to free the closed clasp 24. As a result, the holding force between the clasp 24 and the tissue overcomes any frictional force between the clasp 24 and the PFO closure catheter 14 when the PFO closure catheter 14 is retracted proximally, and the clasp 24 remains in place at the PFO 2, as shown in FIG. 25. The splay posts 188 are moved out of the way of the clips 26 of the clasp 24, such as by moving the splay arm 40 relative to the housing 20 such that the ear or ears 46 of the splay arm 40 engage an aperture, ramp or other feature in the housing 20, where as a result of that engagement at least part of the splay arm 40, and thereby the splay posts 44, are moved out of the path of the clasp 24, freeing it to move. Alternately, the splay posts 188 may be moved out of the path of the clasp 24 in any other suitable manner. Alternately, the splay posts 44 remain substantially fixed, and the clasp 24 has a path out of the clasp applicator 16 that does not intersect the splay posts 44. At least one anvil 48 is also moved out of the way of the clips 26 in any suitable manner. As one example, referring also to FIG. 8, two or more rods 50 may extend within the clasp applicator 16, where at least one anvil 48 is positioned at or near the distal end of a rod 50, and a collar 52 may encircle two or more rods 50 at a location proximal to the anvils 48. The anvils 48 may be moved out of plane with the clips 26 by moving the collar 52 distally, causing the distal ends of the rods 50 and thus the anvils 48 to move closer to one another. At least the distal end of the rods 50 then may be moved in a different direction, such that the anvils 48 do not interfere with the backbone 28 of the clasp 24 as the clasp 24 exits the clasp applicator 18. Any other structure may be used to move at least one anvil 48 out of the path of a corresponding clip 26; where one anvil 48 is moved out of that path, the clasp 24 as a whole may be moved out of contact with the other anvil 48 such as by the splay arm 40 or other structure or mechanism.

Alternately, the clasp 24 may be actively ejected from the clasp applicator 16. Any structures or mechanisms holding the clasp 24 in the clasp applicator 16 are moved or actuated to free the closed clasp 24, and the closed clasp 24 is pushed out of the clasp applicator 16. Where the clasp 24 is frangibly connected to the driver 38, force is exerted on the clasp 24 after it has been closed and its connection tested, in order to separate the clasp 24 from the driver 38. Such force on the clasp 24 may be provided in any suitable manner.

Where the clasp applicator 16 has splayed at least one clip 26 beyond ninety degrees, that amount of splaying facilitates contact between the distal end of the tines 30 of that clip 26 and the tissue of the wall of a tunnel-like PFO 2. As a result, the tines 30 enter that tissue, grasp it, and pull opposed walls of the tunnel-like PFO 2 into closer contact with one another. Such closure may be sufficient to completely close the PFO 2. However, such closure may decrease the diameter of the tunnel-like PFO 2 to a point where a second clasp 24 may be utilized to fully close the PFO 2. If additional clasps 24 are to be used to close the PFO 2 for any reason, the PFO closure catheter 14 may be withdrawn from the patient, and a new PFO closure catheter 14 may be inserted into the patient via the guidewire 56. Alternately, the clasp applicator 16 may be configured to deploy more than one clasp 24 into tissue.

Next, the anchors 54 are collapsed from the second, expanded configuration back to the first, narrower configuration. The anchors 54 may be collapsed simultaneously, or at different times. This collapse may be performed in a manner opposite to which the anchors 54 were previously expanded. For example, the compressive force applied to a basket 60 may be released, allowing the basket 60 to collapse back to its first, narrower configuration. Alternately, a tensile force may be applied to the basket 60 to return it to its first, narrower configuration, such as where at least part of the basket 60 is plastically deformable. As another example, where the anchor 54 is a harpoon 80, the control wire 86 is advanced further distally within the harpoon 80, such that engagement between each barb 90 and the distal edge of each aperture 88 pushes each barb 90 back toward the control wire 86 and then into the lumen 84 of the harpoon 80 in the withdrawal configuration. In this way, the barbs 90 release tissue, allowing the harpoon 80 to be withdrawn proximally out of tissue. Alternately, the barbs 90 may release tissue in any other suitable manner. As one example, the barbs 90 may be configured to reenter the lumen 84 of the harpoon 80 when the control wire 86 is moved proximally. Where the basket 60 is configured as shown in FIG. 12, the guidewire 56 may be released, allowing the basket 60 to return to its initial configuration. Alternately, the guidewire 56 may be moved distally relative to the distal end of the jacket 76 in order to deform the basket 60 back to its initial configuration. If suction has been applied by the catheter 14 to tissue in proximity to the PFO 2, then application of that suction may cease in order to allow the catheter 14 to be moved away from tissue.

The collapsed anchors 54 are then pulled through the closed PFO 2, such as by retracting the guidewire or guidewires 56. In the collapsed configuration, each anchor 54 is small enough to pass through the space between an end of the PFO 2 and the closest clasp 24 and/or through the space between adjacent clasps 24. The anchors 54 are retracted into the clasp applicator 16. Alternately, at least part of at least one anchor 54 need not be retracted into the clasp applicator 16, in whole or in part. The PFO 2 may be inspected under fluoroscopy or any other suitable imaging system to assess whether the PFO 2 has been closed. If not, another clasp 24 may be deployed, in substantially the same manner as set forth above. The guiding catheter 92 is then removed from the patient, as is the PFO closure system 12, in any suitable order. Alternately, one or more of the anchors 54 can be left in the PFO 2 as an implant. In such a case, at least one of the anchors 54 may assist in the closure of the PFO 2.

While the invention has been described in detail, it will be apparent to one skilled in the art that various changes and modifications can be made and equivalents employed, without departing from the present invention. It is to be understood that the invention is not limited to the details of construction, the arrangements of components, the process of closing the PFO 2, and/or the sequence of operations for closing the PFO 2 set forth in the above description or illustrated in the drawings. Exemplary steps set forth in the method for closing the PFO 2 may be omitted as the practitioner sees fit. Further, the invention is not limited to the closure of a PFO 2. Other heart defects, such as atrial septal defects (ASDs), or other tissue defects or openings in other parts of the vasculature or any other part of the body, may be closed utilizing the present invention. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the following claims and their legal equivalents.

What is claimed is:

1. A tool for closing an opening in tissue, comprising:
a catheter;
a clasp applicator positioned substantially at the distal end of said catheter; and
at least one clasp held by said clasp applicator, wherein said clasp includes a plurality of tines and is plastically deformable, and wherein said clasp is plastically splayable by said clasp applicator.

2. The tool of claim 1, incorporating by reference all of the limitations of claim 1, wherein said clasp comprises at least two clips, each fixed to a backbone, each said clip comprising at least two said tines.

3. The tool of claim 1, incorporating by reference all of the limitations of claim 1, wherein said clasp comprises at least two clips, each fixed to the other substantially perpendicularly.

4. The tool of claim 1, incorporating by reference all of the limitations of claim 1, wherein said clasp includes at least three said tines.

5. The tool of claim 1, incorporating by reference all of the limitations of claim 1, further comprising at least one radially-expandable anchor connected to said clasp applicator.

6. The tool of claim 5, incorporating by reference all of the limitations of claim 5, wherein at least one said anchor is a basket that includes a plurality of proximal struts and a plurality of distal struts positioned distal to said proximal struts, and wherein said distal struts are configured to deform outward from the longitudinal centerline of said basket before said proximal struts in response to a compressive force applied to said basket.

7. The tool of claim 5, incorporating by reference all of the limitations of claim 5, further comprising a guidewire, wherein said anchor is connected to said guidewire and wherein said guidewire is slideably received in said clasp applicator.

8. The tool of claim 5, incorporating by reference all of the limitations of claim 5, wherein at least one said anchor is a harpoon.

9. The tool of claim 8, incorporating by reference all of the limitations of claim 8, wherein said harpoon includes a barb both extendable and retractable therefrom.

10. The tool of claim 1, incorporating by reference all of the limitations of claim 1, further comprising a guidewire; wherein said catheter is a rapid exchange catheter slidable along said guidewire.

11. The tool of claim 1, incorporating by reference all of the limitations of claim 1, wherein at least one tine is splayable beyond ninety degrees.

12. A tool for closing an opening in tissue, comprising:
a catheter;
a clasp applicator positioned substantially at the distal end of said catheter;
at least one clasp held by said clasp applicator, wherein said clasp is deployable by said clasp applicator to close the opening in tissue; and
at least one anchor extending distally from said catheter, each said anchor selectively expandable from a first diameter to a second, larger diameter and selectively collapsible from said second diameter to said first diameter, each said anchor movable through the opening in tissue and expandable on the opposite side of the opening in tissue from said clasp applicator.

13. The tool of claim 12, incorporating by reference all of the limitations of claim 12, wherein said anchor comprises a guidewire and a plurality of wires twisted about said guidewire; further comprising an atraumatic tip attached to and extending distally from each said wire and guidewire.

14. The tool of claim 12, incorporating by reference all of the limitations of claim 12, wherein said anchor is a basket, said basket comprising a plurality of struts expandable outward from the longitudinal centerline of said basket.

15. A tool for closing an opening in tissue, comprising:
a catheter;
a clasp applicator positioned substantially at the distal end of said catheter; and
at least one clasp held by said clasp applicator; and
at least one harpoon connected to and deployable from said clasp applicator, said at least one harpoon configured to engage tissue in proximity to the opening in tissue.

16. The tool of claim 15, incorporating by reference all of the limitations of claim 15, wherein at least one said harpoon is movable from position within said clasp applicator to a position extending at least partially from said clasp applicator.

17. The tool of claim 15, incorporating by reference all of the limitations of claim 15, wherein at least one said harpoon has apertures defined therein; further comprising barbs selectively deployable through said apertures.

18. A method for closing a patent foramen ovale in heart tissue of a patient, the patient having a vasculature, comprising:
providing at least one clasp and a clasp applicator that holds said at least one said clasp, wherein said clasp includes a plurality of tines and is plastically deformable;
advancing said clasp applicator through the vasculature to a location in proximity to the patent foramen ovale;
anchoring said clasp applicator with respect to the patent foramen ovale;
splaying said clasp, wherein said splaying plastically deforms said clasp;
advancing said clasp into contact with tissue;
closing said clasp, wherein said closing plastically deforms said clasp; and
removing said clasp applicator from the heart.

19. The method of claim 18, incorporating by reference all of the limitations of claim 18, further comprising at least one anchor extendable distal to said clasp applicator, wherein said anchoring comprises
placing said at least one anchor on a different side of the patent foramen ovale as said clasp applicator, and
expanding said anchor radially outward after said placing.

20. The method of claim 18, incorporating by reference all of the limitations of claim 18, wherein said anchoring comprises extending at least one harpoon from said clasp applicator into heart tissue in proximity to the patent foramen ovale.

21. The method of claim 18, incorporating by reference all of the limitations of claim 18, wherein said anchoring includes applying suction to heart tissue in proximity to the patent foramen ovale.

22. The method of claim 18, incorporating by reference all of the limitations of claim 18, further comprising registering said clasp applicator to the patent foramen ovale after said anchoring.

23. The method of claim 18, incorporating by reference all of the limitations of claim 18, wherein said clasp applicator is positioned at the distal end of a catheter, and wherein said advancing said clasp applicator includes advancing said catheter.

24. The method of claim 18, incorporating by reference all of the limitations of claim 18, wherein said splaying comprises splaying at least one said tine beyond ninety degrees.

25. A method for closing a patent foramen ovale in heart tissue of a patient, the patient having a vasculature, comprising:
providing a clasp having a plurality of tines;
splaying said tines, wherein said splaying plastically deforms said clasp; and
closing said tines into heart tissue in proximity to the patent foramen ovale, wherein said closing plastically deforms said clasp and substantially closes said patent foramen ovale.

26. A method for closing a patent foramen ovale in heart tissue of a patient, the patient having a vasculature, comprising:
providing at least one clasp, and a clasp applicator that holds at least one said clasp;
advancing said clasp applicator through the vasculature to a location in proximity to the patent foramen ovale;
anchoring said clasp applicator with respect to the patent foramen ovale;
closing at least one said clip into heart tissue; and
moving said clasp applicator away from the patent foramen ovale.

27. The method of claim 26, incorporating by reference all of the limitations of claim 26, further comprising providing at least one anchor extendable distally from said clasp applicator; wherein said anchoring comprises moving at least one said anchor through the patent foramen ovale and expanding at least one said anchor on the other side of the patent foramen ovale from said clasp applicator.
